(19) United States
(12) Patent Application Publication
(10) Pub. No.: US 2005/0079614 A1
(43) Pub. Date: Apr. 14, 2005

(54) RNAs able to modulate chromatin silencing

(76) Inventors: Brenda J. Reinhart, Palo Alto, CA (US); David Bartel, Brookline, MA (US)

Correspondence Address: WOLF GREENFIELD & SACKS, PC;FEDERAL RESERVE PLAZA, 600 ATLANTIC AVENUE, BOSTON, MA, 02210-2211, US

(21) Appl. No.: 10/895,644

(22) Filed: Jul. 21, 2004

Related U.S. Application Data
(60) Provisional application No. 60/489,272, filed on Jul. 21, 2003.

(51) Int. Cl. A61K 48/00; C12N 15/85

(52) U.S. Cl. 435/455; 514/44

(57) ABSTRACT

This invention generally relates to nucleotide sequences and, in particular, to nucleotide sequences able to bind to or otherwise associate with DNA or chromatin, or otherwise modulate chromatin silencing. In certain embodiments, the nucleotide sequence may be present in (or encode for) a noncoding and/or nonexpressable RNA having less than 50 or 100 nucleotides, preferably about 20-30 nucleotides. In some cases, a precursor nucleic acid may be cleaved in some fashion to produce the nucleotide sequence. In one set of embodiments, the nucleotide sequences of the invention are not native to the cell, i.e., not normally present in the cell. In certain cases, the nucleotide sequence may be a 20-25 nucleotide RNA molecule that occurs naturally in other cells and/or in other organisms, or the nucleotide sequence may be an artificially generated nucleotide sequence, and in such cases, the nucleotide sequence is referred to herein as “heterochromatic small interfering RNA,” or “heterochromatic siRNA.” The nucleotide sequences may also be present within a nucleic-acid protein complex in certain embodiments. The nucleotide sequences of the invention may inhibit gene function, for example, by interacting with chromatin, and/or by transcriptionally inhibiting mRNA synthesis. In certain cases, the heterochromatic siRNA of the invention can further posttranscriptionally inhibit gene function, e.g., by binding to mRNA. In some cases, inhibition of the gene may be epigenetic or may otherwise be stable for relatively long periods of time, i.e., chromatin may be epigenetically altered through interaction with the nucleotide sequence, for example, through altered methylation of DNA or histones.

### RELATED APPLICATIONS

This application claims the benefit of U.S. Provisional Patent Application Ser. No. 60/489,272, filed Jul. 21, 2003, entitled “RNAs Able to Modulate Chromatin Silencing,” by B. J. Reinhart, et al. This application is incorporated herein by reference.

### BACKGROUND

Small interfering RNAs (siRNAs) and microRNAs (miRNAs) are two types of ˜22-nucleotide (nt) noncoding RNAs that can play important roles as regulators of gene expression in eukaryotes. siRNAs derive from the successive cleavage of long double-stranded RNA (dsRNA). siRNAs direct the destruction of corresponding mRNA targets during RNA interference in animals and during other RNA-silencing phenomena, including posttranscriptional gene silencing of plants and quelling of Neurospora. miRNAs are processed from endogenous hairpin transcripts such that a single miRNA is usually produced from one arm of each hairpin molecule. Certain Caenorhabditis elegans miRNAs are known to direct translational repression of mRNA targets needed for proper larval development, and numerous plant and animal miRNAs are thought to play similar roles in other contexts by targeting mRNAs for translation, attenuation, or destruction. The ribonuclease III protein Dicer is usually required for the processing of both siRNAs and miRNAs from their respective precursor RNAs, and Argonaute (PAZ/PIWI domain) proteins, whose biochemical functions are unclear, are also usually necessary for production or function of both miRNAs and siRNAs.

### SUMMARY OF INVENTION

This invention generally relates to nucleotide sequences and, in particular, to nucleotide sequences able to modulate chromatin silencing. The subject matter of this invention involves, in some cases, interrelated products, alternative solutions to a particular problem, and/or a plurality of different uses of a single system or article.

In one aspect, the invention is a method. In one set of embodiments, the method includes a step of inserting, into a cell, RNA comprising a binding region able to transcriptionally inhibit an expressable gene, where the RNA has less than about 100 nucleotides. In another set of embodiments, the method includes a step of inserting, into a cell, a nucleotide sequence able to cause the cell to produce RNA comprising a nucleotide sequence able to bind to chromatin within the cell to cause chromatin silencing. The method, in yet another set of embodiments, includes a step of inserting, into a cell, RNA able to inhibit an expressable gene for a time greater than an average half-life that the RNA remains intact within the cell. In still another set of embodiments, the method is defined, at least in part, by a step of inserting, into a cell, RNA able to transcriptionally inhibit an expressable gene, where the RNA has less than about 100 nucleotides.

In one set of embodiments, the method includes a step of inserting, into a cell, RNA able to inhibit an expressable gene to create an inhibited gene that, after at least three successive cell divisions of the cell into a plurality of daughter cells, remains inhibited in at least one of the daughter cells. The method is defined, in yet another set of embodiments, by a step of inserting, into a cell, RNA able to inhibit an expressable gene for a time greater than an average half-life that the RNA remains intact within the cell.

The invention is a composition in another aspect. In one set of embodiments, the composition includes isolated RNA comprising a binding region able to transcriptionally inhibit an expressable gene, where the RNA has less than about 100 nucleotides. In another set of embodiments, the composition includes isolated RNA comprising a nucleotide sequence able to bind to chromatin within a cell to cause chromatin silencing, where the RNA has less than about 100 nucleotides. The composition, in yet another set of embodiments, includes isolated RNA able to inhibit an expressable gene within a cell for a time greater than an average half-life that the RNA is able to remain intact within the cell.

In one set of embodiments, the composition includes isolated RNA able to transcriptionally inhibit an expressable gene. The composition, in another set of embodiments, includes isolated RNA able to inhibit an expressable gene within a cell to create an inhibited gene that, after at least three successive cell divisions of the cell into a plurality of daughter cells, remains inhibited in at least one of the daughter cells.

In another aspect, the present invention is directed to a method of making one or more of the embodiments described herein, for example, a heterochromatic siRNA sequence, as further described below. In yet another aspect, the present invention is directed to a method of using one or more of the embodiments described herein, for example, a heterochromatic siRNA sequence, as further described below.

Other advantages and novel features of the invention will become apparent from the following detailed description of various non-limiting embodiments of the invention when considered in conjunction with the accompanying figures. In cases where the present specification and a document incorporated by reference include conflicting disclosure, the present specification shall control.

### BRIEF DESCRIPTION OF THE SEQUENCES

SEQ ID NO: 1 is GAGGCUUUCGGUUUAGUCGC, an RNA sequence arising from Schizosaccharomyces pombe;

SEQ ID NO: 2 is AAUGCGGAGUAAGGCUAAUCACGGUA, an RNA sequence arising from Schizosaccharomyces pombe;

SEQ ID NO: 3 is UCUAGCUUCGCCAUCAAUAAGUA, an RNA sequence arising from Schizosaccharomyces pombe;

SEQ ID NO: 4 is UGGAUUAAGGAGAAGCGGUA, an RNA sequence arising from Schizosaccharomyces pombe;

SEQ ID NO: 5 is ACAAGUGAUAAGAGUAGGUGU, an RNA sequence arising from Schizosaccharomyces pombe;

SEQ ID NO: 6 is UGCGCAACUCCUGCUUAUCGUC, an RNA sequence arising from Schizosaccharomyces pombe;

SEQ ID NO: 7 is UACAAGAUAUAGCGCCACACU, an RNA sequence arising from Schizosaccharomyces pombe;

SEQ ID NO: 8 is UGAGCAUAUCCUAAUGACAGUA, an RNA sequence arising from Schizosaccharomyces pombe;

SEQ ID NO: 9 is UGCCUAUUUAUACAUUUCCC, an RNA sequence arising from Schizosaccharomyces pombe;

SEQ ID NO: 10 is UCUACCUCAGCAGUCCUUGGGAAA, an RNA sequence arising from Schizosaccharomyces pombe;

SEQ ID NO: 11 is UGUGUCCAUAUCCAUGCUGUGUCCA, an RNA sequence arising from Schizosaccharomyces pombe; and

SEQ ID NO: 12 is UAAACAACUUGCAAUAUCUGCCA, an RNA sequence arising from Schizosaccharomyces pombe.

### BRIEF DESCRIPTION OF DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For the purposes of clarity, not every component is labeled in every figure, nor is every component of each embodiment of the invention shown where illustration is not necessary to allow those of ordinary skill in the art to understand the invention. In the figures:

FIGS. 1A and 1B illustrate certain heterochromatic siRNAs in accordance with an embodiment of the invention.

### DETAILED DESCRIPTION

This invention generally relates to nucleotide sequences and, in particular, to nucleotide sequences able to bind to or otherwise associate with DNA or chromatin, or otherwise modulate chromatin silencing. In certain embodiments, the nucleotide sequence may be present in (or encode for) a noncoding and/or nonexpressable RNA having less than 50 or 100 nucleotides, preferably about 20-30 nucleotides. In some cases, a precursor nucleic acid may be cleaved in some fashion to produce the nucleotide sequence. In one set of embodiments, the nucleotide sequences of the invention are not native to the cell, i.e., not normally present in the cell. In certain cases, the nucleotide sequence may be a 20-25 nucleotide RNA molecule that occurs naturally in other cells and/or in other organisms, or the nucleotide sequence may be an artificially generated nucleotide sequence, and in such cases, the nucleotide sequence is referred to herein as “heterochromatic small interfering RNA,” or “heterochromatic siRNA.” The nucleotide sequences may also be present within a nucleic-acid protein complex in certain embodiments. The nucleotide sequences of the invention may inhibit gene function, for example, by interacting with chromatin, and/or by transcriptionally inhibiting mRNA synthesis. In certain cases, the heterochromatic siRNA of the invention can further posttranscriptionally inhibit gene function, e.g., by binding to mRNA. In some cases, inhibition of the gene may be epigenetic or may otherwise be stable for relatively long periods of time, i.e., chromatin may be epigenetically altered through interaction with the nucleotide sequence, for example, through altered methylation of DNA or histones.

The following references are incorporated herein by reference in their entirety: B. J. Reinhart and D. P. Bartel, “Small RNAs Correspond to Centromere Heterochromatic Repeats,” Science, 297:1831 (2002), and U.S. Provisional Patent Application Ser. No. 60/489,272, filed Jul. 21, 2003, entitled “RNAs Able to Modulate Chromatin Silencing,” by B. J. Reinhart, et al.

As used herein, the term “sample” is used in its broadest sense. A sample can originate from a cell or tissue, e.g., from an animal or a plant, or a specimen or culture obtained from any source, as well as biological and environmental samples. Biological samples may be obtained from plants or animals and may encompass fluids, solids, tissues, cells, and gases. These examples are not to be construed as limiting the sample types applicable to the present invention.

The term “cell,” as used herein, is given its ordinary meaning as used in biology. The cell may be an isolated cell, a cell aggregate, or a cell found in a cell culture, in a tissue construct containing cells, or the like. In some cases, the cell may be a genetically engineered cell. As used herein the term “transgenic” when used in reference to a cell (i.e., a “transgenic cell”) refers to a cell that contains at least one heterologous gene. A “heterologous” sequence, as used herein, is an identifiable sequence of nucleotides within a larger nucleic acid molecule that is not found in association with the larger nucleic acid molecule in nature. In contrast, a “homologous” sequence is an identifiable sequence of nucleotides within a larger nucleic acid molecule that is found in association with the larger nucleic acid molecule in nature.

Examples of cells include, but are not limited to, a bacterium or other single-cell organism, a eukaryotic cell, a plant cell, or an animal cell. If the cell is an animal cell, the cell may be, for example, an invertebrate cell (e.g., a cell from a fruit fly), a fish cell (e.g., a zebrafish cell), an amphibian cell (e.g., a frog cell), a reptile cell, a bird cell, or a human or non-human mammal, such as a monkey, ape, cow, sheep, big-horn sheep, goat, buffalo, antelope, oxen, horse, donkey, mule, deer, elk, caribou, water buffalo, camel, llama, alpaca, rabbit, pig, mouse, rat, guinea pig, hamster, dog, or cat. If the cell is from a multicellular organism, the cell may be from any part of the organism. For instance, if the cell is from an animal, the cell may be a cardiac cell, a fibroblast, a keratinocyte, a heptaocyte, a chondracyte, a neural cell, a osteocyte, a muscle cell, a blood cell, an endothelial cell, an immune cell (e.g., a T-cell, a B-cell, a macrophage, a neutrophil, a basophil, a mast cell, an eosinophil), a stem cell, etc. Other cells include those from the bladder, brain, esophagus, fallopian tube, intestines, gallbladder, kidney, liver, lung, ovaries, pancreas, prostate, spinal cord, spleen, stomach, testes, thymus, thyroid, trachea, ureter, urethra, or uterus. Other examples of cells include differentiated cells, such as epithelial cells, epidermal cells, hematopoietic cells, melanocytes, erythrocytes, macrophages, monocytes, or germ line cells such as oocytes and sperm cells; and undifferentiated cells, such as embryonic, mesenchymal, or adult stem cells. The term “germ line cells” refers to cells in the organism which can trace their eventual cell lineage to either the male or female reproductive cells of the organism. Other cells that are referred to as “somatic cells” are cells which do not directly give rise to gamete or germ line cells. Somatic cells, however, also may be used in some embodiments of the invention.

As used herein, “epigenetics” and “epigenetic inheritance” refers to the transmission of information from a cell to its descendants (e.g., daughter cells) without that information being encoded in the nucleotide sequence of the genes. The cell may be any cell, including germ line cells and somatic cells. Epigenetic inheritance may occur in the development of multicellular organisms; dividing fibroblasts, for example, give rise to new fibroblasts even though their genome is identical to that of all other cells. Epigenetic transmission of traits may also occur from one generation to the next in some organisms (i.e., epigenetically transferred through germ line cells). Epigenetic inheritance systems allow cells of different phenotype but identical genotype to transmit their phenotype to their descendants, even in cases where phenotype-inducing stimuli are absent. One example of an epigenetic inheritance system is a chromatin-marking system, where proteins or chemical groups attached to or otherwise associated with DNA can modify its activity. These groups can be copied during DNA replication. Specific examples of such chromatin-marking systems include methylated cytosines and methylated histones.

An “isolated” molecule, as used herein, is a molecule that is free of other substances with which it is ordinarily found in nature or in in vivo systems to an extent practical and appropriate for its intended use. In particular, the isolated molecular species may be sufficiently free from other biological constituents of host cells, or if the species is expressed in host cells, the isolated molecular species may be free of the form or context in which the species ordinarily found in nature. For instance, a nucleic acid encoding a heterochromatic siRNA sequence may ordinarily be found in a host cell in the context of the host cell genomic DNA; however, an isolated nucleic acid encoding the heterochromatic siRNA sequence can be delivered to the host cell, and is thus not found in the same context of the host genomic DNA as the host cell. Alternatively, an isolated nucleic acid can be removed from the host cell or be present in a host cell that does not ordinarily have such a nucleic acid present. Because an isolated molecular species of the invention may be admixed in a preparation with carriers or other agents, e.g., in a pharmaceutical preparation or in cell media, the isolated molecular species may comprise only a small percentage by weight of the preparation. The isolated molecular species is nonetheless substantially pure in that it has been substantially separated from substances with which it may have been associated with in the living system.

As used herein, the term “nucleic acid” is used to mean one or more nucleotides, i.e. a molecule comprising a sugar (e.g. ribose or deoxyribose) linked to a phosphate group and to an exchangeable organic base, which may be a substituted pyrimidine (e.g. cytosine (C), thymidine (T) or uracil (U)) or a substituted purine (e.g. adenine (A) or guanine (G)). The term “nucleic acid” also includes “polynucleotides” or “oligonucleotides,” as those terms are ordinarily used in the art, i.e., polymers of nucleotides, where oligonucleotides are generally shorter in length than polynucleotides. A sequence of nucleotides bonded together, i.e., within a polynucleotide or an oligonucleotide can be referred to as a “nucleotide sequence.” The term “nucleic acid” also includes nucleosides and polynucleosides (i.e. a nucleotide/polynucleotide without the phosphate). Purines and pyrimidines include, but are not limited to, natural nucleosides (for example, adenosine, thymidine, guanosine, cytidine, uridine, deoxyadenosine, deoxythymidine, deoxyguanosine and deoxycytidine), nucleoside analogs (for example, 2-aminoadenosine, 2-thiothymidine, inosine, pyrrolopyrimidine, 3-methyladenosine, C5-bromouridine, C5-fluorouridine, C5-iodouridine, C5-propynyluridine, C5-propynylcytidine, C5-methylcytidine, 7-deazaadenosine, 7-deazaguanosine, 8-oxoadenosine, 8-oxoguanosine, O6-methylguanosine, 2-thiocytidine, 2-aminopurine, 2-amino-6-chloropurine, 2,6-diaminopurine, hypoxanthine), chemically or biologically modified bases (for example, methylated bases), intercalated bases, modified sugars (2′-fluororibose, arabinose, or hexose), modified phosphate groups (for example, phosphorothioates or 5′-N-phosphoramidite linkages), and other naturally and non-naturally occurring nucleobases, including substituted and unsubstituted aromatic moieties. Other such modifications are well-known to those of skill in the art.

As used herein, a first sequence that is “substantially complementary” to a second sequence is one which at least 75% of the first and second sequences are complementary and/or the sequences have a maximum of 1 base mismatch. In some embodiments, the two sequences may be at least 80%, 85%, 90%, 95%, 96%, 97%, 98%, 99%, or 100% complementary. In other embodiments, the two sequences may include a maximum of 0, 1, 2, 3, or 4 mismatches.

A “microRNA” or “miRNA,” as used herein, refers to a RNA molecule derived from genomic loci which is processed from transcripts that can form local RNA hairpin precursor miRNA structures. The terms are used consistently with their common meaning in the art. The mature form of miRNA is a 20-nt to 24-nt species that is usually detectable on Northern blots, and miRNA has the potential to pair to flanking genomic sequences, placing the mature miRNA within an imperfect RNA duplex thought to be needed for its processing from a longer precursor transcript. In addition, miRNAs are typically derived from a segment of the genome that is distinct from predicted protein-coding regions. Thus far, >150 tiny RNAs that satisfy these criteria have been identified in animals.

A “small interfering RNA” or “siRNA,” as used herein, refers to a RNA molecule derived from the successive cleavage of long double-stranded RNA (dsRNA) within a cell to produce an RNA molecule generally have a length of between 15 and 30 nucleotides, and more often between 20 and 25 nucleotides. siRNAs direct the destruction of corresponding mRNA targets during RNA interference in animals, and during other RNA-silencing phenomena, including posttranscriptional gene silencing of plants and quelling of Neurospora.

Similarly, “heterochromatic small interfering RNA,” or “heterochromatic siRNA,” as used herein, also refers to RNA molecules generally having a length of between 15 and 30 nucleotides, and more often between 20 and 25 nucleotides. Heterochromatic siRNA is able to interact with or otherwise associate with DNA, especially chromatin (unlike siRNA, which does not normally interact with chromatin). In some cases, the heterochromatic siRNA is able to bind to or otherwise associate with one or more heterochromatin regions of the chromosome; in other cases, the heterochromatic siRNA is able to bind to or otherwise associate with one or more euchromatin regions of the chromosome; and in some cases, the heterochromatic siRNA is able to bind to or otherwise associate with both heterochromatin and euchromatin regions.

For example, the heterochromatic siRNA, upon interaction with chromatin, may direct the modification of chromatin (i.e., DNA and/or histones associated with the DNA), which may then cause silencing or inhibition of one or more genes within the chromatin. Without wishing to be bound to any theory, it is believed that the heterochromatic siRNA, in the context of a protein-nucleic acid complex comprising one or more proteins and one or more nucleic acids, may pair to (“bind”) or otherwise associate with DNA within the chromatin. The complex of proteins associated with the heterochromatic siRNA may include, for example, but is not limited to, the Argonaute family of proteins. The association of the heterochromatic siRNA complex with DNA may cause methylation of histones adjacent to the DNA, and/or DNA methylation, for example, by directing or guiding methylating enzymes (e.g., methyl transferases) or other protein complexes to the chromatin. In some cases, the methylation or other modification of the DNA and/or histones may then cause silencing of one or more genes, and/or condensation of chromatin. In certain embodiments, gene silencing may occur through the interaction of the heterochromatic siRNA with regulatory regions of the gene, for example, through interaction with transcription factors, promoters, enhancers, and the like. In some cases, heterochromatic siRNA can also bind to mRNA or otherwise further posttranscriptionally inhibit gene function, e.g., in the manner of siRNA. The heterochromatic siRNA sequence may be naturally occurring or artificial. As used herein, an “artificial heterochromatic siRNA,” in reference to a target cell or cell type, is a heterochromatic siRNA that is not native to the cell or cell type, or otherwise is not normally present within the cell (i.e., expressed).

The present invention generally relates to nucleotide sequences able to target DNA or histones, or otherwise modulate chromatin silencing. In some cases, the nucleotide sequences may be a 20-25 nucleotide RNA molecule referred to above as “heterochromatic small interfering RNA,” or “heterochromatic siRNA.” In other cases, the nucleotide sequence is formed when a precursor nucleic acid is processed in some manner (for example, cleaved and/or spliced), to produce the nucleotide sequence. In still other cases, a gene can be transfected into a cell that cause the cell to produce the nucleotide sequence or precursor nucleotide sequence. Delivery of a nucleic acid (or a complex comprising the nucleic acid) containing and/or encoding for the nucleotide sequence to a cell (for example, to transcriptionally inhibit a centromere or a gene) can be effected in any suitable manner known to those of skill in the art (for example, through colloidal dispersion systems or transformation vectors), and can be enhanced in any manner (for example, through the use of a promoter). After delivery, inhibition of the gene and/or the centromere may be epigenetic or may otherwise be stable for relatively long periods of time.

In one aspect, the invention includes a nucleic acid comprising a nucleotide sequence able to interact with DNA in some fashion, for example to transcriptionally inhibit DNA. In one set of embodiments, the nucleotide sequence may be present within the nucleic acid (for example, RNA), optionally in combination with proteins, vectors, other nucleic acid elements or sequences, etc. In another set of embodiments, the nucleic acid may be an RNA molecule consisting essentially of the nucleotide sequence. In one embodiment of the invention, the nucleotide sequence may be part of a nucleic acid, which, in turn, is part of a protein-nucleic acid complex.

In some cases, the nucleic acid may bind to or otherwise associate with DNA to cause inhibition of a gene or a centromere. A “binding region” of a nucleic acid, as used herein, is the portion or nucleotide sequence within the nucleic acid that interacts with and/or specifically, non-covalently causes association of the nucleic acid with DNA or chromatin. It should be noted that the “binding” of the nucleic acid to the DNA or chromatin may be direct or indirect. For example, a nucleic acid may directly bind to DNA or chromatin; a portion of a protein-nucleic acid complex may bind to DNA or chromatin (e.g., the nucleic acid may be complexed to a protein that, in turn, directly or indirectly binds to the DNA); a nucleic acid may bind to a protein associated with the DNA or chromatin, etc. In some cases, such as in heterochromatic siRNA, the binding region of the nucleic acid or the protein-nucleic acid complex may be the entire nucleic acid.

In one set of embodiments, the nucleic acid containing the nucleotide sequence may transcriptionally inhibit DNA. As used herein, a nucleic acid that is able to “transcriptionally inhibit” DNA means that the nucleic acid is able to inhibit DNA transcription, e.g., by binding to or otherwise interacting with DNA or chromatin, such as previously described. For example, if the nucleic acid is heterochromatic siRNA, the nucleic acid may be able to interact with DNA to cause gene or chromatin silencing. In certain cases, such transcriptional inhibition may occur in addition to posttranscriptional inhibition of the nucleic acid, e.g., in the manner of siRNA.

In one preferred set of embodiments, the nucleic acid (or nucleic acid complex) containing the nucleotide sequence is heterochromatic siRNA, as previously described. Heterochromatic siRNAs are able to interact with chromosomes, for example, to cause inhibition of chromatin function and/or gene expression. The heterochromatic siRNA may bind to or otherwise associate with specific regions within the chromatin in some cases, for example, causing methylation and/or other alterations to the DNA and/or histones, etc. The heterochromatic siRNA may thus define a binding region within the nucleic acid according to certain embodiments of the invention. In some cases, gene silencing may also occur as a result of chromatin silencing.

In certain embodiments, the nucleotide sequence (i.e., as part of a nucleic acid) may have an intracellular concentration (e.g., through delivery or expression by the cell) at a level sufficient to cause at least a 2-fold reduction in the expression of one or more proteins. In other embodiments, the nucleotide sequence may be expressed at a level sufficient to cause at least a 2, 5, 15, 20, 25, 30, 35, 40, 45, 50, 55, 60, 65, 70, 75, or 100 fold reduction in the expression of the protein(s). The “fold reduction” may be assessed using any parameter for assessing a quantitative value of protein expression. For instance, a quantitative value can be determined using a label (e.g. fluorescent or radioactive) linked to an antibody. The value is a relative value that can be compared to a control and/or a known value. The level of expression of a target protein(s) may be assessed using routine methods known to those of skill in the art. For instance, a protein may be isolated from a cell and quantified using Western blot analysis or other comparable methodologies, optionally in comparison to a control. Protein levels may also be assessed in a cell using reporter systems or fluorescently labeled antibodies.

Different nucleotide sequences of the invention may have different intracellular concentrations and/or activities, and thus can have different effects on protein expression. For instance, in some cases the nucleotide sequence may be expressed within a nucleic acid at a high level or concentration, and/or may be very efficient such that the transcription of the gene or protein is completely or near completely blocked. In other instances, the expression of the protein may be only reduced slightly over the level that would ordinarily be expressed in that cell at that time under those conditions, in the absence of the expressed nucleotide sequence. Complete inhibition of the expression of the gene or protein is not generally essential. In many cases, partial or low inhibition of protein expression may produce a preferred phenotype. The actual amount that is useful will depend on the particular cell type, the stage of differentiation, conditions to which the cell is exposed, the modulation of other target proteins, the intracellular concentration of the nucleic acid, etc.

In one aspect, the nucleotide sequence may epigenetically affect the chromosome, and/or one or more genes within the chromosome. For example, the nucleotide sequence may cause or otherwise induce methylation of the histones or DNA within the chromatin, and/or the formation of condensed heterochromatin, as previously discussed. Other mechanisms of inhibition are also possible. The epigenetic changes by the nucleotide sequence to the chromosome may persist within the cell even after the nucleotide sequence has been removed (e.g., through degradation, un-binding or dissociation of the nucleic acid containing the nucleotide sequence from the chromosome, etc). Thus, the effects of the inhibition of the gene or chromosome by the nucleotide sequence may persist for a length of time greater than the half-life that the nucleotide sequence is able to remain intact within the cell, and in some cases, greater than two, three, or four or more half-lives. In some cases, the epigenetic modifications caused by the nucleotide sequence may be relatively permanent (e.g., the epigenetic modifications do not substantially decrease with time or with cell divisions, etc.). For instance, the epigenetic modifications may continue within daughter cells of the cell after mitosis/meiosis, and in some cases, for at least two generations of daughter cells, at least three generations of daughter cells, at least four generations of daughter cells, etc.

In one aspect, a nucleic acid (or nucleic acid complex) containing the nucleotide sequence able to target the chromatin can be formed when a precursor nucleic acid is processed in some manner (for example, cleaved and/or spliced), to produce the nucleotide sequence. As used herein, a “precursor nucleic acid” is composed of any type of nucleic acid-based molecule capable of accommodating or incorporating a heterochromatic siRNA sequence or other targeting nucleotide sequence. The precursor nucleic acid may have greater than about 20 and less than about 200, less than about 100, or less than about 50 nucleotides, where some of the nucleotides can be cleaved off the precursor nucleic acid to produce the final nucleotide sequence. In one embodiment, the precursor nucleic acid is a long double-stranded RNA (dsRNA).

Thus, for example, a precursor nucleic acid (or precursor nucleic acid complex) can be delivered to a cell, then processed within the cell into a final nucleic acid containing the nucleotide sequence, for example, a heterochromatic siRNA. Non-limiting examples of precursor nucleic acids and the individual components of the precursor nucleic acids are provided herein; however, the invention is not limited to the examples provided. The nucleotide sequence(s) of the precursor and its components (e.g., as further discussed below) may vary widely. The precursor nucleic acid may be processed in vivo, ex vivo, or in vitro to produce the final nucleotide sequence. For instance, after delivery, the precursor nucleic acid may be cleaved (e.g., by an enzyme within a cell) to produce a heterochromatic siRNA sequence. As an example, the precursor nucleic acid may be processed in a cell by a ribonuclease enzyme. Non-limiting examples of ribonuclease enzymes which are able to process precursor molecules include the RNase II ribonucleases Dicer and Argonaute, as well as RNA-dependent RNA polymerase.

The precursor nucleic acid may also include other components in certain embodiments of the invention, for example, heterologous or homologous stem-loop or other nucleic acid sequence components, or proteins, for example, as previously described. A heterologous precursor nucleic acid may be produced by replacing a portion of a precursor nucleic acid sequence taken from a cell with a sequence substantially complementary to a gene, etc., to be inhibited.

In another aspect of the invention, the nucleotide sequence able to target or otherwise modulate chromatin is delivered into a cell, for example, to inhibit a gene. The nucleotide sequence may be present within a nucleic acid (or precursor nucleic acid), or a gene may be transfected into the cell that causes the cell to produce the target nucleotide sequence (for example, a gene that causes the cell to produce heterochromatic siRNA). Any method or delivery system may be used for the delivery and/or transfection of the nucleic acid in the cell, for example, but not limited to particle gun technology, colloidal dispersion systems, electroporation, vectors, and the like.

In its broadest sense, a “delivery system,” as used herein, is any vehicle capable of facilitating delivery of a nucleic acid (or nucleic acid complex) to a cell and/or uptake of the nucleic acid by the cell. Other example delivery systems that can be used to facilitate uptake by a cell of the nucleic acid include calcium phosphate and other chemical mediators of intracellular transport, microinjection compositions, and homologous recombination compositions (e.g., for integrating a gene into a preselected location within the chromosome of the cell).

The term “transfection,” as used herein, refers to the introduction of a nucleic acid into a cell. Transfection may be accomplished by a variety of means known to the art. Such methods include, but are not limited to, Agrobacterium-mediated transformation (e.g., Komari et al., Curr. Opin. Plant Biol., 1:161 (1998)), particle bombardment mediated transformation (e.g., Finer et al., Curr. Top. Microbiol. Immunol., 240:59 (1999)), protoplast electroporation (e.g., Bates, Methods Mol. Biol., 111:359 (1999)), viral infection (e.g., Porta and Lomonossoff, Mol. Biotechnol. 5:209 (1996)), microinjection, electroporation, and liposome injection. Standard molecular biology techniques are common in the art (See e.g., Sambrook, J. et al., Molecular Cloning: A Laboratory Manual, 2^{nd} ed., Cold Spring Harbor Laboratory Press, New York (1989)).

For instance, in one set of embodiments, genetic material may be introduced into a cell using particle gun technology, also called microprojectile or microparticle bombardment, which involves the use of high velocity accelerated particles. In this method, small, high-density particles (microprojectiles) are accelerated to high velocity in conjunction with a larger, powder-fired macroprojectile in a particle gun apparatus. The microprojectiles have sufficient momentum to penetrate cell walls and membranes, and can carry DNA or other nucleic acids into the interiors of bombarded cells. It has been demonstrated that such microprojectiles can enter cells without causing death of the cells, and that they can effectively deliver foreign genetic material into intact tissue.

In another set of embodiments, a colloidal dispersion system may be used to facilitate delivery of the nucleic acid (or nucleic acid complex) into the cell. As used herein, a “colloidal dispersion system” refers to a natural or synthetic molecule, other than those derived from bacteriological or viral sources, capable of delivering to and releasing the nucleic acid to the cell. Colloidal dispersion systems include, but are not limited to, macromolecular complexes, beads, and lipid-based systems including oil-in-water emulsions, micelles, mixed micelles, and liposomes. One example of a colloidal dispersion system is a liposome. Liposomes are artificial membrane vessels. It has been shown that large unilamellar vessels (“LUV”), which range in size from 0.2 to 4.0 microns can encapsulate large macromolecules within the aqueous interior and these macromolecules can be delivered to cells in a biologically active form (Fraley, et al., Trends Biochem. Sci., 6:77 (1981)).

Lipid formulations for transfection and/or intracellular delivery of nucleic acids are commercially available, for instance, from QIAGEN, for example as EFFECTENE® (a non-liposomal lipid with a special DNA condensing enhancer) and SUPER-FECT® (a novel acting dendrimeric technology) as well as Gibco BRL, for example, as LIPOFECTIN® and LIPOFECTACE®, which are formed of cationic lipids such as N-[1-(2,3-dioleyloxy)-propyl]-N,N,N-trimethylammonium chloride (DOTMA) and dimethyl dioctadecylammonium bromide (DDAB). Methods for making liposomes are well known in the art and have been described in many publications. Liposomes were described in a review article by Gregoriadis, G., Trends in Biotechnology 3:235-241 (1985), which is hereby incorporated by reference.

Electroporation may be used, in another set of embodiments, to deliver a nucleic acid (or nucleic acid complex) to the cell. Electroporation, as used herein, is the application of electricity to a cell in such a way as to cause delivery of the nucleic acid into the cell without killing the cell. Typically, electroporation includes the application of one or more electrical voltage “pulses” having relatively short durations (usually less than 1 second, and often on the scale of milliseconds or microseconds) to a media containing the cells. The electrical pulses typically facilitate the non-lethal transport of extracellular nucleic acids into the cells. The exact electroporation protocols (such as the number of pulses, duration of pulses, pulse waveforms, etc.), will depend on factors such as the cell type, the cell media, the number of cells, the substance(s) to be delivered, etc., and can be determined by one of ordinary skill in the art.

In yet another set of embodiments, the nucleic acid may be delivered to the cell in a vector. In its broadest sense, a “vector” is any vehicle capable of facilitating the transfer of the nucleic acid to the cell such that the nucleic acid can be processed and/or expressed in the cell. Preferably, the vector transports the nucleic acid to the cells with reduced degradation, relative to the extent of degradation that would result in the absence of the vector. The vector optionally includes gene expression sequences or other components able to enhance expression of the nucleic acid within the cell. The invention also encompasses the cells transfected with these vectors. Host cells include, for instance, cells and cell lines, e.g. prokaryotic cells (e.g., E. coli) and eukaryotic cells (e.g., dendritic cells, CHO cells, COS cells, yeast expression systems, and recombinant baculovirus expression in insect cells). Other cells have been previously described.

In general, vectors useful in the invention include, but are not limited to, plasmids, phagemids, viruses, other vehicles derived from viral or bacterial sources that have been manipulated by the insertion or incorporation of the nucleotide sequence (or precursor nucleic acid) of the invention. Viral vectors useful in certain embodiments include, but are not limited to, nucleic acid sequences from the following viruses: retroviruses such as Moloney murine leukemia viruses, Harvey murine sarcoma viruses, murine mammary tumor viruses, and Rouse sarcoma viruses; adenovirus, or other adeno-associated viruses; SV40-type viruses; polyoma viruses; Epstein-Barr viruses; papilloma viruses; herpes virus; vaccinia virus; polio viruses; and RNA viruses such as retroviruses. One can readily employ other vectors not named but known to the art.

Some viral vectors can be based on non-cytopathic eukaryotic viruses in which non-essential genes have been replaced with the nucleotide sequence of interest. Non-cytopathic viruses include retroviruses, the life cycle of which involves reverse transcription of genomic viral RNA into DNA with subsequent proviral integration into host cellular DNA.

Genetically altered retroviral expression vectors may have general utility for the high-efficiency transduction of nucleic acids. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the cells with viral particles) can be found in Kriegler, M., Gene Transfer and Expression, A Laboratory Manual, W. H. Freeman Co., New York (1990) and Murry, E. J. Ed., Methods in Molecular Biology, Vol. 7, Humana Press, Inc., Cliffton, N.J. (1991), both hereby incorporated by reference.

Another example of a virus for certain applications is the adeno-associated virus, which is a double-stranded DNA virus. The adeno-associated virus can be engineered to be replication-deficient and is capable of infecting a wide range of cell types and species. The adeno-associated virus further has advantages, such as heat and lipid solvent stability; high transduction frequencies in cells of diverse lineages, including hemopoietic cells; and/or lack of superinfection inhibition, which may allow multiple series of transductions.

Another vector suitable for use with the invention is a plasmid vector. Plasmid vectors have been extensively described in the art and are well-known to those of skill in the art. See e.g., Sambrook, et al., Molecular Cloning: A Laboratory Manual, Second Edition, Cold Spring Harbor Laboratory Press, 1989. These plasmids may have a promoter compatible with the host cell, and the plasmids can express a peptide from a gene operatively encoded within the plasmid. Some commonly used plasmids include pBR322, pUC18, pUC19, pRC/CMV, SV40, and pBlueScript. Other plasmids are well-known to those of ordinary skill in the art. Additionally, plasmids may be custom-designed, for example, using restriction enzymes and ligation reactions, to remove and add specific fragments of DNA or other nucleic acids, as necessary. The present invention also includes vectors for producing nucleic acids or precursor nucleic acids containing a desired nucleotide sequence (which can, for instance, then be cleaved or otherwise processed within the cell to produce heterochromatic siRNAs). These vectors may include a sequence encoding a nucleic acid and an in vivo expression element, as further described below. In some cases, the in vivo expression element includes at least one promoter.

The nucleic acid, in one embodiment, may be operably linked to a gene expression sequence which directs the expression of the nucleic acid within the cell (e.g., to produce a heterochromatic siRNA or a precursor to the heterochromatic siRNA). The nucleic acid sequence and the gene expression sequence are said to be “operably linked” when they are covalently linked in such a way as to place the transcription of the nucleic acid sequence under the influence or control of the gene expression sequence. A “gene expression sequence,” as used herein, is any regulatory nucleotide sequence, such as a promoter sequence or promoter-enhancer combination, which facilitates the efficient transcription and translation of the nucleotide sequence to which it is operably linked. The gene expression sequence may, for example, be a eukaryotic promoter or a viral promoter, such as a constitutive or inducible promoter. Promoters and enhancers consist of short arrays of DNA sequences that interact specifically with cellular proteins involved in transcription, for instance, as discussed in Maniatis, T. et al., Science 236:1237 (1987), incorporated herein by reference. Promoter and enhancer elements have been isolated from a variety of eukaryotic sources including genes in plant, yeast, insect and mammalian cells and viruses (analogous control elements, i.e., promoters, are also found in prokaryotes).

The selection of a particular promoter and enhancer depends on what cell type is to be used and the mode of delivery. For example, a wide variety of promoters have been isolated from plants and animals, which are functional not only in the cellular source of the promoter, but also in numerous other plant and/or animal species. There are also other promoters (e.g., viral and Ti-plasmid) which can be used. For example, these promoters include promoters from the Ti-plasmid, such as the octopine synthase promoter, the nopaline synthase promoter, the mannopine synthase promoter, and promoters from other open reading frames in the T-DNA, such as ORF7, etc. Promoters isolated from plant viruses include the 35S promoter from cauliflower mosaic virus (CaMV). Promoters that have been isolated and reported for use in plants include ribulose-1,3-biphosphate carboxylase small subunit promoter, phaseolin promoter, etc.

Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of Moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

Thus, a variety of promoters and regulatory elements may be used in the expression vectors of the present invention. For example, in some preferred embodiments an inducible promoter is used to allow control of nucleic acid expression through the presentation of external stimuli (e.g., environmentally inducible promoters). Thus, the timing and amount of nucleic acid expression may be controlled. Non-limiting examples of expression systems, promoters, inducible promoters, environmentally inducible promoters, and enhancers are described in International Patent Application Publications WO 00/12714, WO 00/11175, WO 00/12713, WO 00/03012, WO 00/03017, WO 00/01832, WO 99/50428, WO 99/46976 and U.S. Pat. Nos. 6,028,250, 5,959,176, 5,907,086, 5,898,096, 5,824,857, 5,744,334, 5,689,044, and 5,612,472 each of which is herein incorporated by reference in its entirety.

As used herein, an “expression element” can be any regulatory nucleotide sequence, such as a promoter sequence or promoter-enhancer combination, which facilitates the efficient expression of the nucleic acid. The expression element may, for example, be a mammalian or viral promoter, such as a constitutive or inducible promoter. Constitutive mammalian promoters include, but are not limited to, polymerase promoters as well as the promoters for the following genes: hypoxanthine phosphoribosyl transferase (HPTR), adenosine deaminase, pyruvate kinase, and alpha-actin. Exemplary viral promoters which function constitutively in eukaryotic cells include, for example, promoters from the simian virus, papilloma virus, adenovirus, human immunodeficiency virus (HIV), Rous sarcoma virus, cytomegalovirus, the long terminal repeats (LTR) of Moloney leukemia virus and other retroviruses, and the thymidine kinase promoter of herpes simplex virus. Other constitutive promoters are known to those of ordinary skill in the art. Promoters useful as expression elements of the invention also include inducible promoters. Inducible promoters are expressed in the presence of an inducing agent. For example, a metallothionein promoter can be induced to promote transcription in the presence of certain metal ions. Other inducible promoters are known to those of ordinary skill in the art. The in vivo expression element can include, as necessary, 5′ non-transcribing and 5′ non-translating sequences involved with the initiation of transcription, and can optionally include enhancer sequences or upstream activator sequences.

Using any gene transfer technique, such as the above-listed techniques, an expression vector harboring the nucleic acid may be transformed into a cell to achieve temporary or prolonged expression. Any suitable expression system may be used, so long as it is capable of undergoing transformation and expressing of the precursor nucleic acid in the cell. In one embodiment, a pET vector (Novagen, Madison, Wis.), or a pBI vector (Clontech, Palo Alto, Calif.) is used as the expression vector. In some embodiments an expression vector further encoding a green fluorescent protein (GFP) is used to allow simple selection of transfected cells and to monitor expression levels. Non-limiting examples of such vectors include Clontech's “Living Colors Vectors” pEYFP and pEYFP-C 1.

In some cases, a selectable marker may be included with the nucleic acid being delivered. As used herein, the term “selectable marker” refers to the use of a gene that encodes an enzymatic or other detectable activity (e.g., luminescence or fluorescence) that confers the ability to grow in medium lacking what would otherwise be an essential nutrient. A selectable marker may also confer resistance to an antibiotic or drug upon the cell in which the selectable marker is expressed. Selectable markers may be “dominant” in some cases; a dominant selectable marker encodes an enzymatic or other activity (e.g., luminescence or fluorescence) that can be detected in any cell or cell line.

The following examples are intended to illustrate certain aspects of certain embodiments of the present invention, but do not exemplify the full scope of the invention.

### EXAMPLE

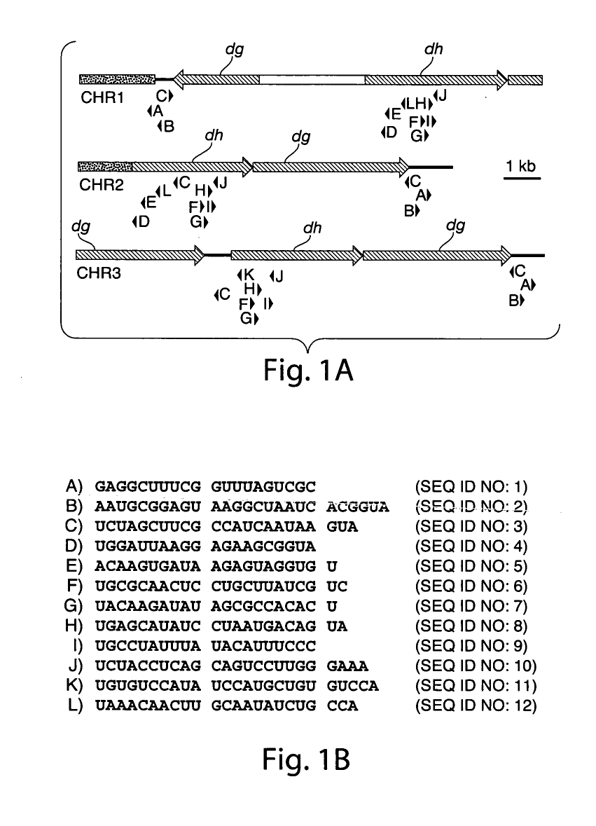

A Dicer and an Argonaute homolog were previously reported in the genome of Schizosaccharomyces pombe (a fission yeast), implying that certain RNAs might play an important role in fission yeast gene regulation. To investigate this possibility, in this example, endogenous RNAs were closed from exponentially growing S. pombe using a method designed to clone RNAs with the features of Dicer cleavage products, i.e., ˜22-nt RNAs with 5′-phosphate and 3′-hydroxyl groups. Of the 61 products detected, sequenced and cloned, 49 clones were found to be fragments of degraded rRNA or tRNA, which are typically seen as background in such cloning efforts. Each of the remaining 12 cloned sequences matched the S. pombe centromeric repeats (FIG. 1). These products were sequenced as follows: GAGGCUUUCGGUUUAGUCGC (SEQ ID NO: 1) (“Sequence A”), AAUGCGGAGUAAGGCUAAUCACGGUA (SEQ ID NO: 2) (“Sequence B”), UCUAGCUUCGCCAUCAAUAAGUA (SEQ ID NO: 3) (“Sequence C”), UGGAUUAAGGAGAAGCGGUA (SEQ ID NO: 4) (“Sequence D”), ACAAGUGAUAAGAGUAGGUGU (SEQ ID NO: 5) (“Sequence E”), UGCGCAACUCCUGCUUAUCGUC (SEQ ID NO: 6) (“Sequence F”), UACAAGAUAUAGCGCCACACU (SEQ ID NO: 7) (“Sequence G”), UGAGCAUAUCCUAAUGACAGUA (SEQ ID NO: 8) (“Sequence H”), UGCCUAUUUAUACAUUUCCC (SEQ ID NO: 9) (“Sequence I”), UCUACCUCAGCAGUCCUUGGGAAA (SEQ ID NO: 10) (“Sequence J”), UGUGUCCAUAUCCAUGCUGUGUCCA (SEQ ID NO: 11) (“Sequence K”), and UAAACAACUUGCAAUAUCUGCCA (SEQ ID NO: 12) (“Sequence L”). The loci and orientation of matches to representative repeats are indicated below each centromere fragment in FIG. 1. Sequence K only matched Chr3, and Sequences D, E and L matched other repeats on Chr3, as shown in FIG. 1. The centromeric repeats shown in FIG. 1 include dg and dh. Although the innerrnost centromeric repeats contained tRNA sequences, all tRNA fragments that were cloned also mapped elsewhere.

The majority of the centromeric RNAs were found to be from the dh repeat, an element that can confer heterochromatic silencing on another locus and may be sufficient for centromere function along with the centromeric central core. None of the other RNAs detected match other heterochromatic regions, such as the centromeric dg repeat, the centromeric core sequences, or the mating type locus region homologous to the dh repeat.

Because the S. pombe centromeres are large regions (40 to 100 kb) with homologous repeating units, these small RNAs may have arisen from a single domain of one centromere or from multiple sites on different chromosomes. These small RNAs do not appear to be miRNAs, in that transcription of adjacent genomic sequence would not produce foldback structures akin to those of the miRNA precursors. Instead, the small RNAs are generally suggestive of siRNAs, corresponding to transcripts generated from both DNA strands of the repeat region (FIG. 1). Most of the centromeric small RNAs cluster within or near these transcripts, which suggests that RNAs produced from each strand of the repeat are able to anneal to form dsRNA that is cleavable by Dicer into the small RNAs. Mutations in dcr1 and ago1 in S. pombe can reduce centromeric repeat H3 K9 methylation, which is necessary for centromere function. Accordingly, these small RNAs may be referred to as “heterochromatic siRNAs,” and may specify epigenetic modification.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of” “only one of,” or “exactly one of.” “Consisting essentially of”, when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A method, comprising:
inserting, into a cell, RNA comprising a binding region able to transcriptionally inhibit an expressable gene, wherein the RNA has less than about 100 nucleotides.

2. The method of claim 1, wherein the RNA has less than about 50 nucleotides.

3. The method of claim 1, wherein the RNA has between about 20 nucleotides and about 25 nucleotides.

4-8. (canceled)

9. The method of claim 1, wherein the RNA includes a heterochromatic siRNA sequence.

10. The method of claim 1, wherein the RNA is able to bind to a chromosome.

11. The method of claim 10, wherein the RNA is able to bind to a euchromatic region within the chromosome.

12. The method of claim 1, wherein the binding region comprises a nucleotide sequence able to bind to chromatin to cause chromatin silencing.

13. The method of claim 1, wherein the binding region is between about 20 nucleotides and about 25 nucleotides in length.

14. A composition, comprising:
isolated RNA comprising a binding region able to transcriptionally inhibit an expressable gene, wherein the RNA has less than about 100 nucleotides.

15-19. (canceled)

20. The composition of claim 14, wherein the RNA includes a heterochromatic siRNA sequence.

21-22. (canceled)

23. The composition of claim 14, wherein the binding region comprises a nucleotide sequence able to bind to chromatin to cause chromatin silencing.

24. (canceled)

25. A composition, comprising:
isolated RNA comprising a nucleotide sequence able to bind to chromatin within a cell to cause chromatin silencing, wherein the RNA has less than about 100 nucleotides.

26-29. (canceled)

30. The composition of claim 25, wherein the RNA includes a heterochromatic siRNA sequence.

31-32. (canceled)

33. A method, comprising:
inserting, into a cell, a nucleotide sequence able to cause the cell to produce RNA comprising a nucleotide sequence able to bind to chromatin within the cell to cause chromatin silencing.

22. (canceled)

34. The method of claim 33, wherein the nucleotide sequence is able to cause the cell to produce RNA able to bind to a euchromatic region within a chromosome within the cell.

35. The method of claim 33, wherein the nucleotide sequence is able to cause the cell to produce a heterochromatic siRNA precursor.

36. (canceled)

37. A composition, comprising:
isolated RNA able to inhibit an expressable gene within a cell for a time greater than an average half-life that the RNA is able to remain intact within the cell.

38. The composition of claim 37, wherein the RNA is able to inhibit the expressable gene for at least three successive cell divisions of the cell into a plurality of daughter cells.

39. A method, comprising:
inserting, into a cell, RNA able to inhibit an expressable gene for a time greater than an average half-life that the RNA remains intact within the cell.

40-43. (canceled)

44. The method of claim 39, wherein the RNA includes a heterochromatic siRNA sequence.

45-47. (canceled)


SEQUENCE LISTING (12 sequences)

SEQ ID NO: 1 - RNA, 20 nt, Schizosaccharomyces pombe
gaggcuuucgguuuagucgc

SEQ ID NO: 2 - RNA, 26 nt, Schizosaccharomyces pombe
aaugcggaguaaggcuaaucacggua

SEQ ID NO: 3 - RNA, 23 nt, Schizosaccharomyces pombe
ucuagcuucgccaucaauaagua

SEQ ID NO: 4 - RNA, 20 nt, Schizosaccharomyces pombe
uggauuaaggagaagcggua

SEQ ID NO: 5 - RNA, 21 nt, Schizosaccharomyces pombe
acaagugauaagaguaggugu

SEQ ID NO: 6 - RNA, 22 nt, Schizosaccharomyces pombe
ugcgcaacuccugcuuaucguc

SEQ ID NO: 7 - RNA, 21 nt, Schizosaccharomyces pombe
uacaagauauagcgccacacu

SEQ ID NO: 8 - RNA, 22 nt, Schizosaccharomyces pombe
ugagcauauccuaaugacagua

SEQ ID NO: 9 - RNA, 20 nt, Schizosaccharomyces pombe
ugccuauuuauacauuuccc

SEQ ID NO: 10 - RNA, 24 nt, Schizosaccharomyces pombe
ucuaccucagcaguccuugggaaa

SEQ ID NO: 11 - RNA, 25 nt, Schizosaccharomyces pombe
uguguccauauccaugcugugucca

SEQ ID NO: 12 - RNA, 23 nt, Schizosaccharomyces pombe
uaaacaacuugcaauaucugcca